(19) United States
(12) Patent Application Publication
(10) Pub. No.: US 2026/0124161 A1
(43) Pub. Date: May 7, 2026

(54) NITISINONE ADMINISTRATION TO LIVESTOCK FOR HEMATOPHAGOUS ARTHROPOD CONTROL

(71) Applicant: Board of Regents of the University of Nebraska, Lincoln, NE (US)

(72) Inventors: Georgina Bingham, Lincoln, NE (US); Roberto Cortinas, Lincoln, NE (US); Sean Avedissian, Lincoln, NE (US)

(21) Appl. No.: 19/378,083

(22) Filed: Nov. 3, 2025

Related U.S. Application Data
(60) Provisional application No. 63/715,261, filed on Nov. 1, 2024.

(51) Int. Cl. A61K 31/122; A61P 33/14

(52) U.S. Cl. CPC A61K 31/122; A61P 33/14

(57) ABSTRACT

The present application provides methods for the control of hematophagous arthropods by administration of nitisinone to animals from which the arthropods feed. Hematophagous arthropods have evolved mechanisms of detoxifying the tyrosine in blood meals. Nitisinone (2-(2-nitro-4-trifluoromethylbenzoyl)-1,3-cyclohexanedione) inhibits the detoxification of tyrosine and leads to the death of hematophagous arthropods. Blood from cattle treated with nitisinone kills blood-feeding arthropods and presents a novel means of protecting cattle from such arthropods.

### CROSS-REFERENCE TO RELATED APPLICATIONS

This patent application claims the benefit of U.S. Provisional Application No. 63/715,261 filed on Nov. 1, 2024, the entire contents of which are incorporated herein by reference.

### FIELD OF INVENTION

The present disclosure relates to methods for controlling hematophagous arthropods by administering nitisinone to subjects, such as livestock.

### BACKGROUND

Blood-feeding arthropods, including flies, ticks, mosquitos and other arthropods, are key vectors in the transmission of zoonotic pathogens. Stable flies pose a large threat to cattle production with an annual estimate of over two billion dollars in economic damage associated with direct feeding on livestock. The impact of blood-feeding on agricultural animal production includes reduced weight gain, decreased milk production, impaired grazing efficiency, overall diminished animal welfare, and increased transmission of disease to domesticated animals and humans. Current approaches to address blood-feeding arthropods include backrubbers, pesticide tags, chemical sprays, pour-ons, and abamectin injectables. However, these methods are limited in controlling arthropod populations for various reasons.

Accordingly, there is a need in the art for novel, targeted approaches to control blood-feeding arthropods. Blood meals require the digestion of a high flux of metabolites, some being highly toxic. Tyrosine, although a minor component of blood meals, becomes toxic at high concentrations. For this reason, blood-feeding arthropods have evolved specialized mechanisms to degrade tyrosine post-ingestion. Thus, the exploration of novel biochemical targets within the tyrosine degradation pathway presents a strategic alternative.

### SUMMARY

A method for controlling hematophagous arthropods in animals is provided. The method comprises administering nitisinone to an animal in an amount effective to cause mortality in hematophagous arthropods that feed on blood from the animal and result in control of the hematophagous arthropods. The animal may be selected from the group consisting of cattle, sheep, goats, horses, swine, poultry, domesticated animals including dogs and cats, poultry or other birds and zoological animals. The hematophagous arthropods may be any blood feeding arthropod such as stable flies (Stomoxys calcitrans), horn flies (Haematobia irritans), tsetse flies (Glossina spp.), ticks (Rhipicephalus, Dermacentor), mosquitoes (Aedes, Culex, and Anopheles spp.), biting midges (Culicidae spp.), and kissing bugs (Triatominae spp.). The nitisinone may be administered via a route selected from orally, via injection, or topically. The effective amount of nitisinone may be from about 0.5 mg/kg to about 2 mg/kg body weight of the animal. The control of the arthropods may be direct killing of the arthropods via the toxicity of the blood meal in the presence of nitisinone. The mortality of the hematophagous arthropods may occur within about 24 to 48 hours after feeding on the blood.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows that topical and oral exposure of NTBC is lethal to stable flies. (A) In oral treatments associated with a citrated (3.7 g sodium citrate/L) blood meal (USDA, Lincoln, NE), a Hemotek feeder (Hemotek, United Kingdom) was utilized to feed the flies for the designated treatment (0.0, 0.0005, 0.00075, 0.001, 0.0015, 0.002, 0.005 mg/mL NTBC) for approximately ten minutes. If any of the flies failed to consume any of the blood, they were removed from the experiment. (B) Topical exposure of NTBC to stable flies was tested by using a Hamilton syringe to apply concentrations of 0 (control), 0.0005, 0.005, 0.01, 0.025, 0.05, and 0.1 mg/mL NTBC (Ambeed, Inc.—Batch No. A384235-011) onto the flies prior to placement in jars). All treatments were fed at 24 hours with 10% sucrose solution using cotton balls in addition to data recording. Data was then repeated at 48 hours (and 72 hours for oral treatment).

FIG. 2 shows the direct effects of NTCB-mediated inhibition of tyrosine degradation on stable flies. Stable flies fed a control (PBS) were compared visually with stable flies fed with NTBC and bovine blood. An Olympus SZX16 microscope (Olympus, Tokyo) was utilized for magnification while the OLYMPUS cellSems Dimension application was used to digitally capture images of the flies. Three images were captured of each fly with a focus on the eyes, abdomen, and body side-profile.

FIG. 3 shows that stable flies fed on blood from intravenous or intramuscular NTBC cattle result in mortality. (A) Intramuscular exposure of nitisinone (NTBC) is lethal to Stable flies. Calf blood was collected from animals treated intramuscularly with NTBC and presented to stable flies using a Hemotek membrane feeder for approximately ten minutes. Flies that failed to feed were excluded. Treatments included positive controls (0.05 mg/mL and 0.0075 mg/mL NTBC) and a PBS negative control. Blood samples were collected on 31 May (IV31MB2-IV31MB9) at 30 min (B2) and hourly (B3-B9) post-dose; and during subsequent sampling on four days following was of a lower frequency of four times per day (IV01LB1-IV04LB4). Percent mortality was measured at 24 h and 48 h. High mortality (60-95%) was observed in early post-dose samples. (B) Intravenous exposure of nitisinone (NTBC) is lethal to stable flies. Calf blood was collected from animals treated intravenously with NTBC and offered to stable flies using a Hemotek membrane feeder (Hemotek, United Kingdom) for approximately ten minutes. Flies that failed to feed were excluded. Treatments included positive controls (0.05 mg/mL and 0.0075 mg/mL NTBC) and a phosphate-buffered saline (PBS) negative control. Blood samples were collected on 31 May (IV31MB2-IV31MB9) at 30 min (B2) and hourly (B3-B9) post-dose; and during subsequent sampling on four days following was of a lower frequency of four times per day (IV01LB1-IV04LB4). Percent mortality was recorded at 24 h and 48 h. The highest mortality (>90%) occurred in early post-dose samples from the 31 May series.

### DETAILED DESCRIPTION

The present invention provides methods for using nitisinone (2-(2-nitro-4-trifluoromethylbenzoyl)-1,3-cyclohexanedione) (also abbreviated as “NTBC”) to control hematophagous arthropods. The NTBC is administered to the animal from which the arthropods will feed, taking on a blood meal. The blood meal in combination with the NTBC results in accumulation of toxic levels of tyrosine metabolites and kills the arthropods. The examples demonstrate the ability to kill stable flies in cattle production. Hematophagous arthropods, which include blood-feeding arthropods such as stable flies, horn flies, mosquitoes, ticks, and other parasitic species, may pose substantial threats to animal and human health and welfare. These arthropods may cause direct harm through blood feeding, stress responses in animals, and transmission of various pathogens and diseases.

Methods for controlling hematophagous arthropods in animals by administering nitisinone (2-(2-nitro-4-trifluoromethylbenzoyl)-1,3-cyclohexanedione) to an animal in an amount effective to cause mortality in hematophagous arthropods that feed on blood from the animal and result in control of the hematophagous arthropods are provided. Nitisinone or NTBC, inhibits 4-hydroxyphenylpyruvate dioxygenase (HPPD) in the hematophagous arthropods that feed on blood from the animal. HPPD is an enzyme involved in the tyrosine degradation pathway. When hematophagous arthropods consume blood meals containing nitisinone, the compound may disrupt the arthropods' ability to metabolize tyrosine causing accumulation of toxic tyrosine metabolites that result in mortality of the hematophagous arthropods after feeding on a blood meal including the nitisinone. While the blood meal is toxic to the arthropods, the NTBC is safe to use in the animals and is being used at levels or concentrations that do not cause deleterious or adverse effects to the animals.

The method may provide a novel approach to pest control that leverages the unique metabolic vulnerabilities of blood-feeding arthropods. Unlike traditional pesticides that may require direct contact or environmental application, the method may transform the treated animal into a self-protecting host. The mortality of hematophagous arthropods may occur within a defined timeframe after the arthropods consume the nitisinone-containing blood meal, providing effective control of arthropod populations. As used herein, to control arthropods refers to a reduction in the number of arthropods, reduction in the speed of growth or reproduction of the arthropods, the causing of deleterious physiological changes, death, lack of reproduction capability, or deviation from normal arthropod phenotype as one with skill in the art would understand.

### Nitisinone

Nitisinone possesses specific physicochemical and pharmacokinetic properties that contribute to the effectiveness of the method for controlling hematophagous arthropods. The compound has a molecular weight of 329.24 g/mol and exhibits moderate lipophilicity. At physiological pH (7.4), NTBC may have aqueous compatibility. The aqueous solubility of nitisinone may range from approximately 0.1 to 10 mg/mL, which may facilitate formulation in various delivery systems. The compound may demonstrate a plasma half-life of approximately 50-60 hours in mammals (from human studies), providing sustained protection against hematophagous arthropods over extended periods.

The NTBC compound demonstrates selective toxicity to blood-feeding arthropods while showing no negative effects on non-blood feeding arthropods such as pollinators. This selectivity may be attributed to the specific metabolic requirements of hematophagous arthropods for tyrosine degradation following blood meal consumption. Non-blood feeding arthropods may not encounter the same metabolic stress from tyrosine accumulation, as these arthropods do not consume blood meals containing high concentrations of tyrosine that require active detoxification pathways. The selective mechanism of action may minimize impacts on beneficial arthropods and other non-target organisms, supporting sustainable pest management practices in agricultural and veterinary applications.

### Animal Applications

The method for controlling hematophagous arthropods may be applied to a wide range of animal species that may be susceptible to blood-feeding arthropod infestations. The animals suitable for treatment with nitisinone may include various domestic, agricultural, and wild species that may benefit from protection against hematophagous arthropods and the diseases these arthropods may transmit. The animal may be selected from the group consisting of cattle, sheep, goats, horses, swine, and zoological animals. Cattle may represent a primary target for treatment, as these animals may be particularly susceptible to stable flies, horn flies, and other blood-feeding arthropods that may cause economic losses in agricultural operations. The Examples focus on cattle, but similar results are expected with other agricultural or domestic animals. The results provided in the Examples demonstrated that there were no adverse effects on feed conversion or digestion in cows after intravenous or intramuscular administration of the NTBC. Similar results would be expected from other ruminants such as goats.

The method may be applied to birds, which may represent another category of animals that may benefit from nitisinone treatment. Birds may be susceptible to various blood-feeding arthropods and may experience health and productivity impacts from these parasites. In some cases, the bird may be selected from the group consisting of chickens, quail, turkeys, ducks, and pigeons.

The method may be applied to animals belonging to the family Canidae, which may include various wild and domestic canine species. In some cases, the animal may be a domesticated dog. Domesticated dogs may be treated with nitisinone to provide protection against mosquitoes, ticks, and other blood-feeding arthropods that may transmit diseases such as heartworm, Lyme disease, and Rocky Mountain spotted fever. The treatment may be particularly beneficial in areas where vector-borne diseases may pose substantial risks to canine health.

Animals belonging to the family Felidae may also be suitable for nitisinone treatment. In some cases, the animal may be a domesticated cat. Domesticated cats may benefit from nitisinone treatment to prevent infestations of fleas, ticks, and mosquitoes that may transmit various feline diseases. The method may provide an alternative approach to traditional flea and tick control products.

The method may be applied to animals of the family Cervidae, which may include various deer species and related ungulates. In some cases, the animal may be a deer or moose. Deer may be treated with nitisinone in managed populations, such as those in wildlife preserves or captive breeding programs, to control blood-feeding arthropods that may impact health and reproduction. The treatment of cervids may also provide indirect benefits by reducing the reservoir of vector-borne diseases that may affect both wildlife and domestic animal populations.

### Target Arthropods

The hematophagous arthropods may include any arthropod that feeds on a blood meal from another animal. Hematophagous arthropods include, but are not limited to, stable flies (Stomoxys calcitrans), horn flies (Haematobia irritans), tsetse flies (Glossina spp.), ticks (Rhipicephalus, Dermacentor), mosquitoes (Aedes, Culex, and Anopheles spp.), biting midges (Culicidae spp.), and kissing bugs (Triatominae spp.). Each of these arthropod types may present distinct challenges to animal health and agricultural productivity, and the method may provide effective control across this diverse range of blood-feeding arthropods.

The Examples demonstrate the effectiveness of the methods provided here on stable fly mortality. These arthropods may cause substantial economic losses in cattle operations. Stable flies may inflict painful bites on livestock, leading to reduced weight gain, decreased milk production, and impaired grazing efficiency. The economic impact of stable flies may exceed two billion dollars annually in the United States alone, with individual cattle potentially losing up to 0.7 pounds per day during peak fly seasons.

Many of these arthropods are known to transmit severe diseases by transmission of viruses, bacteria or parasites while obtaining a blood meal. Several of these diseases are associated with large economic impact in agricultural animals, severe disease and economic impact, many are zoonotic also affecting humans as well. The diseases include Zika virus, West Nile virus, and other encephalitis causing viruses, Lyme disease, trypanosomiasis, and malaria. Control of hematophagous arthropods could result in better control of these infectious diseases as well by limiting the arthropod vectors key to transmission of these infectious agents.

### Routes of Administration

The nitisinone may be administered to animals through various routes that may provide flexibility in treatment protocols and may accommodate different animal management systems. The administration routes may be selected based on factors such as animal species, management practices, treatment duration requirements, and practical considerations for implementation in various settings. The nitisinone may be administered orally, via injection, or topically. The choice of the route of administration will be impacted by the distinct animal management practices used for the various animals and may be impacted by the economic realities and ability to directly handle the different animals to which the nitisinone is being applied, the frequency of administration in the particular animal and other considerations that will be apparent to those of skill in the art.

In one aspect, the administration is by injection, which may be intramuscular or intravenous. Injectable administration routes may provide rapid achievement of therapeutic concentrations and may offer precise dosing control for nitisinone delivery. The intramuscular route may be particularly suitable for animals that may require periodic handling for other management procedures, allowing for integration of nitisinone treatment with existing animal care protocols. The absorption profile from intramuscular injection may provide therapeutic concentrations for periods extending beyond 24 hours, offering effective protection against hematophagous arthropods with reduced frequency of administration. The intravenous route may be suitable for situations requiring immediate protection against hematophagous arthropods or for animals that may require rapid establishment of therapeutic concentrations. The pharmacokinetic profile of intravenous administration may result in higher initial concentrations followed by gradual decline, providing effective arthropod control during peak exposure periods or for arthropods blood feeding pattern is tightly limited to a particular season. The sub-cutaneous injection of a sustained release formulation similar to the ruminal pellet/bolus, or contraceptive pellet may offer extended release.

In another aspect, the administration is topical by application of a spray on, wipe on or a pour on formulation. As used herein, a pour on formulation refers to a liquid-based delivery wherein the solution is poured directly on top of the animals. Spray formulations may be particularly useful in situations where animals may be processed through handling facilities, allowing for rapid treatment of large numbers of animals. The wipe-on approach may be suitable for smaller animals or for situations where precise application may be desired. Wipe-on formulations may offer advantages in terms of reduced waste and improved control over the applied dose compared to spray applications. Pour-on formulations may provide convenient application of nitisinone along the dorsal line of animals, taking advantage of the natural spreading properties of the formulation to achieve broad coverage. Pour-on applications may be particularly suitable for cattle and other large animals where the formulation may spread across large surface areas through natural animal movement and grooming behaviors.

In a further aspect, the administration is oral. Oral administration may represent a practical and widely applicable route for delivering nitisinone to animals. The oral route may provide sustained delivery of the compound through various formulation approaches that may integrate with existing animal feeding and management practices. Oral administration may be accomplished through a mineral lick (such as a salt lick), a rumen delivered pellet, or application directly to feed. For animals that self-clean such as poultry preening or cats bathing themselves, oral administration may be accomplished by application to the feathers or fur. Each of these options offers different advantages for specific animal management systems. As used herein, the mineral lick may be a salt lick. Mineral licks may provide a convenient method for delivering nitisinone to grazing animals such as cattle, sheep, deer, horses and goats. The mineral lick formulation may incorporate nitisinone into a salt or mineral matrix that animals may consume voluntarily as part of their normal feeding behavior. The mineral lick approach may provide sustained, low-dose delivery of nitisinone over extended periods, maintaining therapeutic concentrations in the animal's blood while minimizing handling requirements and labor costs associated with treatment administration. In the Examples, the application of nitisinone was shown to not negatively impact rumen function or feed digestibility in the cattle. Thus feed-based delivery is also an option. The compound may be delivered through supplemented rations that may be formulated to provide specific concentrations of nitisinone based on animal body weight and feeding patterns. Controlled-release boluses may provide another feed-based delivery method for nitisinone administration. The boluses may be designed to remain in the rumen (a rumen pellet) or other digestive compartments for extended periods, gradually releasing nitisinone to maintain therapeutic concentrations. The controlled-release formulation may reduce the frequency of treatment administration and lower the costs associated with administration of the NTBC.

The amount of nitisinone administered may depend on the route of administration. For injection routes as used in the examples, an effective amount of nitisinone for the control of arthropods is from about 0.1 mg/kg to 5 mg/kg, suitably 0.5 mg/kg to about 2 mg/kg body weight of the animal. This dosage range may provide therapeutic concentrations in the animal's blood that may be lethal to blood-feeding arthropods while maintaining safety margins for the treated animals. The effective amount may be determined based on factors including animal species, body weight, administration route, and the specific hematophagous arthropods targeted for control.

The concentration range for topical applications may extend from 0.0005 mg/mL to higher concentrations depending on the specific formulation requirements and target efficacy levels. In some cases, concentrations of 0.005 mg/mL, 0.01 mg/mL, 0.025 mg/mL, 0.05 mg/mL, or 0.1 mg/mL may be utilized for topical applications, with higher concentrations potentially providing more rapid achievement of therapeutic blood levels or extended duration of protection.

The mortality of the hematophagous arthropods may occur within about 24 to 48 hours after feeding on the blood meal including nitisinone. This timeframe may provide effective control of arthropod populations while allowing sufficient time for the compound to exert its metabolic effects within the arthropod tissues. The mortality timeline may be consistent across different species of hematophagous arthropods, including stable flies, horn flies, and other blood-feeding arthropods that may be targeted by the method.

The method may achieve mortality rates exceeding 50%, 60% or even 70% in stable flies within 24 hours following blood meal consumption from treated animals. These high mortality rates may demonstrate the effectiveness of nitisinone in disrupting the tyrosine degradation pathway in blood-feeding arthropods. The mortality rates may increase further at 48 hours post-feeding, with some data showing mortality rates exceeding 85% at this extended timeframe. Thus, the methods provided herein may achieve 75%, 80%, 85% or even higher mortality in arthropods after consumption of a blood meal comprising nitisinone in a short time frame of a few days. The high mortality rates may provide effective population control of hematophagous arthropods in treated animal populations.

The effectiveness of nitisinone in causing arthropod mortality may be accompanied by visible physiological changes in treated arthropods that may serve as indicators of the compound's mechanism of action. These visible effects may provide confirmation of the metabolic disruption caused by nitisinone and may demonstrate the specific targeting of the tyrosine degradation pathway in blood-feeding arthropods. Nitisinone may cause melanin buildup in the eyes of treated arthropods as a visible indicator of effectiveness. The melanin accumulation may result from the disruption of the tyrosine degradation pathway, leading to the conversion of accumulated tyrosine to melanin through alternative metabolic routes. The eyes of treated stable flies may exhibit a dark maroon to nearly black coloration, contrasting with the vibrant red coloration observed in control arthropods that have not consumed nitisinone-containing blood meals. The visible color change may provide immediate feedback to practitioners regarding the success of treatment protocols and may allow for rapid assessment of treatment effectiveness without requiring laboratory analysis or extended observation periods. The method may induce tissue damage and structural deformation in the abdomens of treated arthropods. The abdominal tissue damage may manifest as shriveling and structural deformation that may be distinct from the normal appearance of healthy arthropods. Arthropods showing pronounced melanin buildup and abdominal deformation may be more likely to succumb to the toxic effects of tyrosine accumulation within the 24 to 48-hour mortality timeframe. The progression of visible effects may serve as an early indicator of impending mortality.

The method may demonstrate selectivity for blood-feeding arthropods while maintaining safety in treated animals. Nitisinone may not negatively impact rumen function or feed digestibility in animals receiving treatment. The lack of negative impact on rumen function may be particularly relevant for ruminant animals such as cattle, sheep, and goats that may represent primary targets for nitisinone treatment. The rumen environment may maintain normal microbial populations and fermentation processes despite the presence of nitisinone, allowing for continued efficient digestion of feed materials. The maintenance of normal digestibility may ensure that treated animals may continue to derive full nutritional benefit from their diets while receiving protection against hematophagous arthropods.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference unless explicitly indicated otherwise. The present disclosure shall control in the event there are any disparities between any definitions and/or descriptions found in the cited references.

The following examples are meant only to be illustrative and are not meant as limitations on the scope of the invention or of the appended claims.

### EXAMPLES

### Example 1

In the following example, the inventors describe using NTBC for control of stable flies in cattle production.

Vector-borne diseases account for a significant proportion of global infectious disease burden. The rise of pesticide resistance and concerns over environmental and non-target effects necessitate the development of alternative control strategies. This study investigates the potential of targeting the tyrosine degradation pathway in blood-feeding arthropods using the compound nitisinone (NTBC), originally developed for human metabolic disorders. We assessed NTBC's stability in bovine rumen fluid, its effects on feed digestibility, its lethality to hematophagous arthropods through topical and oral routes, and its pharmacokinetics in bovine models. NTBC demonstrates selective toxicity to blood-feeding arthropods and pharmacological viability, supported by additional pharmacodynamic and toxicological findings. These results suggest promise for NTBC as a component of integrated vector management programs.

### Results:

### Topical and Oral Exposure of NTBC is Lethal to Stable Flies

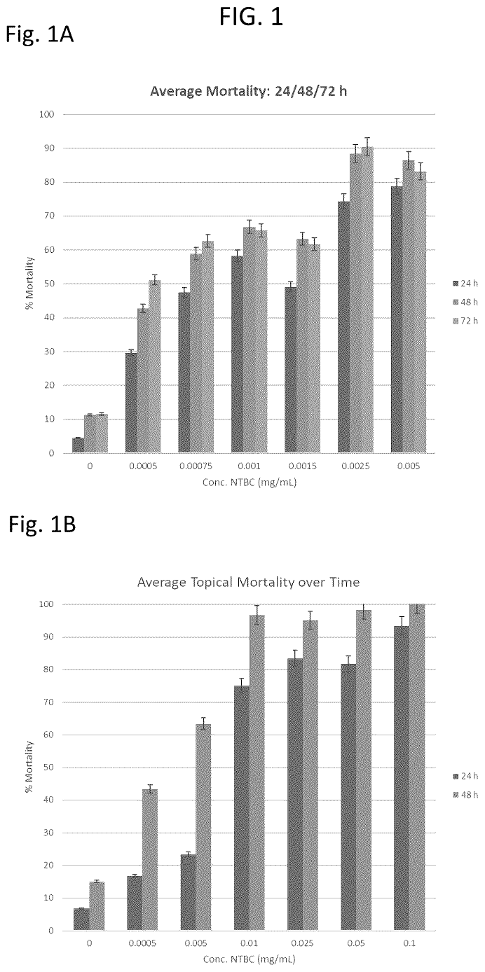

To evaluate the ability of NTBC to cause mortality in stable flies, the chemical was studied through topical approach (FIG. 1B). After a subsequent blood meal, high mortality rates in stable flies were observed with the LD50 is estimated to be at a dose between 0.005 and 0.01 mg/ml. The flies were re-evaluated after 48 hours to observe longer term exposure to the drug and toxic metabolite buildup with the LD50 is estimated to be at a dose between 0.0005 and 0.005 mg/mL. This scenario would occur if NTBC was applied directly onto cattle through sprays or through other methods such as pesticide tags.

Another route of NTBC application that was explored was through oral ingestion of the drug (FIG. 1A). Stable flies fed with different concentrations of NTBC in bovine blood resulted in an LD50 between 0.00075 and 0.001 mg/ml at 24 hours and between 0.0005 and 0.00075 mg/ml at 48 hours post feeding treatment. These results would be consistent with a blood-feeding arthropod taking in NTBC alongside a blood meal such as it being present in bovine blood before feeding. Both topical and oral routes present two different mechanisms to cause stable fly mortality with a blood meal, although at different concentrations of NTBC.

### NTBC Causes Tissue Damage in Stable Flies

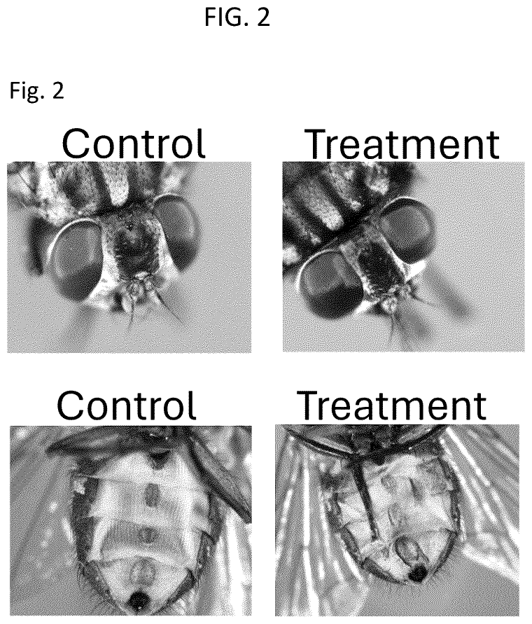

Due to NTBC's role in inhibiting the tyrosine degradation pathway and the ability to cause the buildup of toxic metabolites in an organism, its direct effects on stable flies were explored. Stable flies fed a control (PBS) were compared visually with stable flies fed with NTBC and bovine blood (FIG. 2). Comparing ocular images for the two groups of stable flies, the control group displayed a vibrant, brighter red coloration while the treated group had a dark maroon, nearly black color. This coloration is consistent with melanin buildup in the eyes of the treated stable flies due to blockage of the tyrosine degradation pathway. Looking at the abdomens for each group, there does not appear to be any darkening of the abdominal tissue for either group, but the NTBC treated group has some structural deformation that may indicate reduced functionality of the insect's physiology.

Stable Flies Fed on Blood from Intravenous/Intramuscular NTBC Cattle Result in Mortality

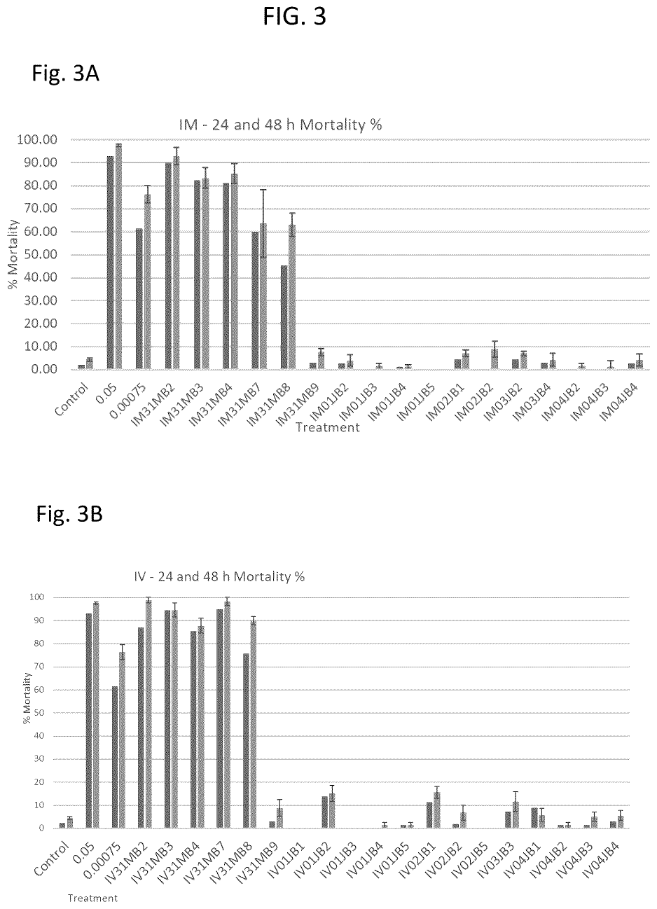

To assess the best method of NTBC introduction into cattle an experiment was designed to compare intramuscular (IM) and intravenous (IV) injections of NTBC into cattle. The cattle then had a series of blood draws to compare stable fly mortality rates after feeding upon the blood. Intravenous injection (FIG. 3B) resulted in a mortality rate of over 70% after 24 hours and over 85% after 48 hours for the blood samples for the first day of sampling. Then for the second through fifth day of sampling, the mortality rate drastically decreased to below 20%. Intramuscular injection (FIG. 3A) displayed a similar dropping pattern in percent mortality in blood samples after the first day. As the blood draws continued within the first day though, the mortality rates for both 24 and 48 hours decreased with later samples. The last blood draw from the first day of sampling (IM31B9) displayed the largest drop in mortality compared to the other samples. Then in the second through fifth days of sampling, the mortality was largely lower than the first day with less than 10% mortality. Significant mortality rates during the first day samples provide the basis for the possibility of implementation of NTBC through these methods.

### Discussion:

### Confirmation of Nitisinone in Mortality Rates and Imaging Support

While NTBC has been analyzed for its effectiveness in other blood-feeding organisms, this study specifically looked at the drug's ability to address stable fly populations that prey on cattle. According to the results, NTBC is indeed an adequate possibility to utilize through multiple outlets to inhibit blood-feeding arthropods. Topical treatment showed sufficient ability at causing stable fly mortality. Oral ingestion of the drug also offers another mechanism to apply the inhibitory treatment. Another benefit of this approach is the lesser quantity of the drug required to achieve similar levels of mortality as the topical treatment. These experiments point to the possibility of NTBC being a useful choice to combat blood-feeding arthropod populations either combined with other pesticides or by itself. This additional method of control provides support for growing threats of pesticide resistance and due to its pinnacle role in blocking the tyrosine breakdown.

### Advancements in Treatment Options Through IV/IM

IV and IM injections of NTBC were selected to conduct the preliminary study based on the rates of mortality in stable flies through oral ingestion of the drug. Both IM and IV injections (FIG. 3) resulted in a significant mortality rate of stable flies for the first day of blood samples after treatment. IM injections had slightly more tapering off in mortality rates as the average mortality dipped slightly under 50% for 24 hours after treatment for the eighth blood draw of the first day. After blood draw four of the first day, the mortality rate started to decline under 80% average mortality after 24 hours for the IM treatment. This trend was not observed in the IV treatment where the average mortality rate was still above 70% mortality for the eighth blood draw of the first day.

### Concluding Remarks

Targeting the tyrosine degradation pathway via NTBC represents a promising direction in vector management. While further optimization is required, particularly in formulation and delivery, preliminary findings validate the feasibility of this novel biochemical intervention. The combined biochemical, pharmacokinetic, and entomological evidence supports the advancement of NTBC as a selective agent.

### Materials and Methods

### Ethics Statement

All animal procedures were conducted in compliance with the ethical standards of the University of Nebraska Institutional Animal Care and Use Committee (IACUC). The experimental protocols involving cattle were reviewed and approved under IACUC Project ID 2417. All efforts were made to minimize animal discomfort during handling and treatment procedures.

### Stable Flies and Stable Fly Treatments

Adult Stomxys calcitrains colonies were obtained at the US Department of Agriculture (USDA), Agricultural Research Service, Agroecosystem Management Research Unit (Lincoln, NE). They were maintained at 22° C., a 12-hour light and dark photoperiod and given 10% sucrose solution ad libitum. The flies were starved from a blood meal 12 hours prior to testing. Stable flies were removed from the cage using a KingFurt bug vacuum catcher (KingFurt) and then anesthetized using either CO₂ or cold temperatures. Stable flies were then separated into individual Mason jars labeled for the individual treatment types.

For topical treatments, the solution was applied using a Hamilton syringe to apply concentrations of 0 (control), 0.0005, 0.005, 0.01, 0.025, 0.05, and 0.1 mg/mL NTBC (Ambeed, Inc.—Batch No. A384235-011) onto the flies prior to placement in the jars. In oral treatments associated with a citrated (3.7 g sodium citrate/L) blood meal (USDA, Lincoln, NE), a Hemotek feeder (Hemotek, United Kingdom) was utilized to feed the flies for the designated treatment (0.0, 0.0005, 0.00075, 0.001, 0.0015, 0.002, 0.005 mg/mL NTBC) for approximately ten minutes. If any of the flies failed to consume any of the blood, they were removed from the experiment. Data was then repeated at 48 hours (and 72 hours for oral treatment).

### Example 2: (Prophetic Example for Application Via Salt Lick)

A salt lick comprising Nitisinone will be made available to cattle, with estimated formulated dose with concentration ranges from approximately 0.5 mg/kg to about 1 mg/kg body weight of the animal per day. This will provide regular ingestion of nitisinone for the control of hematophagous arthropods.

### Example 3: (Prophetic Example for Application Via Injectable Pellets)

An injectable pellet with estimated formulated NTBC dose with concentration ranges from approximately 0.5 mg/kg to about 1 mg/kg body weight of the animal per day.

### Example 4: (Prophetic Example for Application Via Topical “Pour-On” Formulations)

A nitisinone solution will be applied directly onto cattle, with concentrations ranging from 0.0005 to 0.1 mg/mL NTBC.

### Example 5: (Prophetic Example for Application Via Feed-Based Delivery)

A feed treated with formulated NTBC with estimated formulated dose with concentration ranges from approximately 0.5 mg/kg to about 1 mg/kg body weight of the animal per day.

What is claimed:

1. A method for controlling hematophagous arthropods in animals, comprising:
administering nitisinone to an animal in an amount effective to cause mortality in hematophagous arthropods that feed on blood from the animal and result in control of the hematophagous arthropods.

2. The method of claim 1, wherein the animal is selected from the group consisting of cattle, sheep, goats, horses, swine, and zoological animals.

3. The method of claim 1, wherein the animal is a bird.

4. The method of claim 3 wherein the bird is selected from the group consisting of chickens, quail, turkeys, ducks, and pigeons.

5. The method of claim 1, wherein the animal belongs to the family Canidae.

6. The method of claim 5, wherein the animal is a domesticated dog.

7. The method of claim 1, wherein the animal belongs to family Felidae.

8. The method of claim 7, wherein the animal is a domesticated cat.

9. The method of claim 1, wherein the animal is of the family Cervidae.

10. The method of claim 9, wherein the animal is a deer and moose.

11. The method of claim 1, wherein the hematophagous arthropods are selected from the group consisting of stable flies (Stomoxys calcitrans), horn flies (Haematobia irritans), tsetse flies (Glossina spp.), ticks (Rhipicephalus, Dermacentor), mosquitoes (Aedes, Culex, and Anopheles spp.), biting midges (Culicidae spp.), and kissing bugs (Triatominae spp.).

12. The method of claim 1, wherein the nitisinone is administered via a route selected from the group consisting of orally, via injection, or topically.

13. The method of claim 12, wherein the administration is via injection and the injection is intramuscular or intravenous.

14. The method of claim 12, wherein the administration is topical by applying a spray on, wipe on or pour on formulation.

15. The method of claim 12, wherein the administration is oral and through a mineral lick, a rumen delivered pellet or feed.

16. The method of claim 1, wherein the effective amount of nitisinone is from about 0.5 mg/kg to about 2 mg/kg body weight of the animal.

17. The method of claim 1, wherein the mortality of the hematophagous arthropods occurs within about 24 to 48 hours after feeding on the blood.

18. The method of claim 1, wherein the nitisinone does not negatively impact rumen function or feed digestibility in the animal.
